(19) United States
(12) Patent Application Publication
(10) Pub. No.: US 2026/0321435 A1
(43) Pub. Date: Oct. 15, 2026

(54) THREE-DIMENSIONAL CARDIAC STRAIN ANALYSIS METHODS AND SYSTEMS

(71) Applicants: The Regents of the University of California, Oakland, CA (US); Georgia Institute of Technology, Atlanta, GA (US)

(72) Inventors: Satya Prakash PRADHAN, Irvine, CA (US); Mohammad Saber HASHEMI, Irvine, CA (US); Arash KHERADVAR, Irvine, CA (US); Arash YAVARI, Atlanta, GA (US)

(21) Appl. No.: 19/461,361

(22) Filed: Jan. 27, 2026

Related U.S. Application Data
(60) Provisional application No. 63/750,196, filed on Jan. 27, 2025.

(51) Int. Cl. G16H 50/50; A61B 5/055; A61B 6/50; A61B 8/08

(52) U.S. Cl. CPC G16H 50/50; A61B 5/055; A61B 6/503; A61B 8/0883

(57) ABSTRACT

Methods and systems for three-dimensional cardiac strain analysis are disclosed. An example method includes obtaining digital image data of the heart and generating segmented surfaces by segmenting an endocardial surface and an epicardial surface from the digital image data. The method further includes constructing, based on the segmented surfaces, a tetrahedral mesh representation of a myocardium of the heart, and enforcing an incompressibility constraint by refining a geometry of the epicardial surface. Herein, the tetrahedral mesh representation comprises a plurality of tetrahedral elements. The method concludes with computing a strain tensor for each of the plurality of tetrahedral elements at each time step of a cardiac cycle, and generating a visualization of myocardial contraction based on strain tensors computed from the plurality of tetrahedral elements. An example system includes one or more processors configured to implement the above-described method.

### CROSS-REFERENCE TO RELATED APPLICATION

This application claims the benefit of priority to U.S. Provisional Patent Application No. 63/750,196, filed on Jan. 27, 2025. The entire contents of the before-mentioned patent application are incorporated by reference as part of the disclosure of this application.

### STATEMENT REGARDING FEDERALLY SPONSORED RESEARCH

This invention was made with Government support under Grant No. HL153724, awarded by the National Institutes of Health. The Government has certain rights in the invention.

### TECHNICAL FIELD

This patent document generally relates to biomedical imaging and analysis, and more particularly, to cardiac strain imaging and analysis using cine echocardiography.

### BACKGROUND

Understanding myocardial deformation is essential for evaluating cardiac function and diagnosing heart disease. Cardiac strain imaging is an effective noninvasive tool for diagnosing myocardial diseases and monitoring patients. It provides detailed insight into the intrinsic contractility of both the right and left ventricles, helps evaluating diastolic and systolic function, and is useful for identifying subclinical dysfunction.

Echocardiography is the most widely used imaging modality for clinical strain analysis. Strain from cine echocardiography is calculated using speckle-tracking, in which a thin region of interest containing sufficient trackable features—referred to as a kernel—is identified and matched between pairs of frames, and then followed throughout the cardiac cycle to quantify tissue movement. However, strain analysis in cardiology clinics typically reports simplified metrics such as global longitudinal strain, overlooking 3D myocardial deformation.

### SUMMARY

The disclosed technology is related to methods and systems for three-dimensional (3D) cardiac strain analysis using volumetric medical imaging data obtained from, for example, magnetic resonance imaging (MRI), echocardiography (echo), or computed tomography (CT) scans, among others.

In an example aspect, a system in accordance with the disclosed technology extracts position data from both endocardial and epicardial surfaces, thereby creating triangulated surfaces (e.g., a triangular tessellation) with a one-to-one correspondence between vertices on the endocardial and epicardial surfaces. The system then computes, for various time instances during the cardiac cycle, three-dimensional (3D) deformation gradients by partitioning the myocardial volume into tetrahedral elements, calculating strain tensors while enforcing local incompressibility constraints. This approach enables the calculation of all strain components at each location, providing a complete and high-resolution picture of cardiac mechanics that surpasses single-component limitations of current ultrasound platforms. Furthermore, computing all the strain components advantageously enables improved diagnostic accuracy when comparing the strain metrics from a patient to those of a healthy subject and more accurate 3D visualizations.

Embodiments of the disclosed technology offer several advantages over existing systems. Unlike current commercial packages that primarily focus on segmental or limited dimensional strains, the described embodiments, among other features and benefits, provide detailed insights into local tissue deformation patterns through analysis of principal strain directions. The described embodiments can compute different strain measures and identify functional fiber orientation in the myocardial volume, and are adaptable to any volumetric medical imaging modality, making them more versatile than current approaches that are typically optimized for specific imaging technologies. In some embodiments, the enforcement of local incompressibility constraints and the ability to calculate various strain measures (e.g., Cauchy-Green, Euler-Almansi, and Green-Lagrange strains) provides a more comprehensive and accurate analysis of cardiac function compared to existing methods.

In another example aspect, a system for producing 3D strain data associated with a heart is disclosed. The system includes one or more processors configured to obtain measurement data corresponding to a motion of the heart related to a cardiac cycle comprising a plurality of time steps. For a reference time step and a second time step of the plurality of time steps, the one or more processors is configured to generate, based on the measurement data, multiple 3D representations comprising an endocardial surface, a myocardial layer, and an epicardial surface of the heart, and determine, in each of the multiple 3D representations, a first tessellation on the endocardial surface and a second tessellation on the epicardial surface such that each first polygon of the first tessellation corresponds to a respective second polygon of the second tessellation. The one or more processors is further configured to determine, based on the measurement data associated with the reference time step and the second time step, a first deformation metric for a first polyhedron, a second deformation metric for a second polyhedron, and a third deformation metric for third polyhedron, and determine, based on the first deformation metric, the second deformation metric, and the third deformation metric, one or more strain metrics associated with the motion through at least a portion of the cardiac cycle to enable a generation of a 3D visualization of the one or more strain metrics on a representative epicardial or endocardial surface.

In yet another example aspect, a method for producing 3D strain data associated with a heart is disclosed. The method includes obtaining measurement data corresponding to a motion of the heart related to a cardiac cycle comprising a plurality of time steps. Then, for at least two of the plurality of time steps, the method includes generating, based on the measurement data, a plurality of 3D representations comprising an endocardial surface and an epicardial surface of the heart, and determining, in each of the plurality of 3D representations, a first plurality of vertices on the endocardial surface and a second plurality of vertices on the epicardial surface, each of the first plurality of vertices corresponding to a respective vertex of the second plurality of vertices. The method then includes selecting one of the plurality of time steps as a reference time step and another one of the plurality of time steps as a second time step, determining, based on the measurement data associated with the reference time step and the second time step, a deformation gradient corresponding to a volume of a myocardial layer of the heart between a set of the first plurality of vertices and a respective set of the second plurality of vertices, and determining, based on the deformation gradient, one or more strain metrics associated with the motion through at least a portion of the cardiac cycle to enable generation of a 3D visualization of the one or more strain metrics on a representative epicardial surface.

In yet another example aspect, a method for producing 3D strain data associated with a heart is disclosed. The method includes obtaining digital image data of the heart, generating segmented surfaces by segmenting an endocardial surface and an epicardial surface from the digital image data, and constructing, based on the segmented surfaces, a tetrahedral mesh representation of a myocardium of the heart, wherein the tetrahedral mesh representation comprises a plurality of tetrahedral elements. The method then includes enforcing an incompressibility constraint by refining a geometry of the epicardial surface, computing a strain tensor for each of the plurality of tetrahedral elements at each time step of a cardiac cycle, and generating a visualization of myocardial contraction based on strain tensors computed from the plurality of tetrahedral elements.

In yet another example aspect, an apparatus comprising a memory and a processor that implements the above-described methods is disclosed.

In yet another example aspect, the above-described methods may be embodied as processor-executable code and may be stored on a non-transitory computer-readable program medium.

The above and other aspects and features of the disclosed technology are described in greater detail in the drawings, the description and the claims.

### BRIEF DESCRIPTION OF DRAWINGS

FIG. 1 illustrates different types of cardiac strain measurements that can be made on the left ventricle of the heart.

FIGS. 2A and 2B illustrate a side-view and a top-view, respectively, of an example segmentation of the left ventricle.

FIG. 3 illustrates a schematic flowchart showing an overview of an example strain analysis method.

FIG. 4 illustrates a flowchart of an example method of producing three-dimensional (3D) strain data associated with a heart.

FIG. 5 illustrates 4D echocardiography, image processing, and segmentation results for representative LV-focused and RV-focused DICOMs.

FIGS. 6A and 6B illustrate example representative endocardial and epicardial surfaces, respectively, of the left ventricle extracted by the described embodiments.

FIGS. 6C and 6D illustrate example representative endocardial and epicardial surfaces, respectively, of the right ventricle extracted by the described embodiments.

FIGS. 7A and 7B illustrate examples of triangular tessellations on the representative endocardial and epicardial surfaces of the left and right ventricles, respectively.

FIG. 8 illustrates an example of the one-to-one correspondence between the vertices on endocardial and epicardial surfaces.

FIG. 9 illustrates an example transformation of a octahedron element from a reference configuration to a deformed configuration.

FIG. 10A illustrates an example partitioning of a octahedron element into three non-overlapping tetrahedrons with vertices on both epicardial and endocardial surfaces.

FIG. 10B illustrates an example partitioning that results in adjacent octahedrons not interlocking due to a tetrahedral gap.

FIG. 10C illustrates another example partitioning that results in adjacent octahedrons interlocking with no gap between them.

FIG. 11 illustrates an example transformation of a tetrahedral element from a reference configuration to a deformed configuration.

FIG. 12 illustrates an example of visualizing the principal direction of the most contractile principal strain on representative left and right ventricle endocardium models.

FIGS. 13A-13C illustrate example bullseye visualization maps showing fiber strain (or primary contraction) and thickness strain for both the left ventricle (LV) and right ventricle (RV).

FIG. 14 illustrates examples of the orientation of maximum contraction, using the signed angle, using biventricular bullseye visualization maps.

FIGS. 15 and 16 illustrate flowcharts of example methods of producing 3D strain data associated with a heart.

FIG. 17 illustrates a block diagram of an example device that can be configured to implement the disclosed technology.

### DETAILED DESCRIPTION

The heart is a muscular organ that pumps blood through the blood vessels of a body. In humans, the heart is approximately the size of a closed first and is located between the lungs, in the middle compartment of the chest. The heart is divided into four chambers: upper left and right atria and lower left and right ventricles, and is surrounded by a fluid-filled sac called the pericardium, which serves to protect this vital organ. The layers of the heart include:
the epicardium, which is the outer layer of the heart wall and is composed primarily of loose connective tissue, including elastic fibers and adipose tissue. The epicardium functions to protect the inner heart layers, and contains coronary blood vessels, which supply the heart wall with blood.
the myocardium, which is the middle layer of the heart wall. It is composed of cardiac muscle fibers, which enable heart contractions. The myocardium is the thickest layer of the heart wall, with its thickness varying in different parts of the heart. The myocardium of the left ventricle is the thickest, as this ventricle is responsible for generating the power needed to pump oxygenated blood from the heart to the rest of the body.
the endocardium, which is the thin inner layer of the heart wall. This layer lines the inner heart chambers, covers heart valves, and is continuous with the endothelium of large blood vessels. The endocardium of the atria consists of smooth muscle, as well as elastic fibers.

The evaluation of strain during the cardiac cycle is crucial for understanding the structure and function of the heart. For example, stress and strain fields in the myocardium can be used to determine the orientation of myocardial fiber function. While the computation of stress depends on the material properties of a living tissue—that are challenging to determine noninvasively—strain is dependent only on the kinematics of deformation, which can be determined from medical images outputted by different medical imaging modalities.

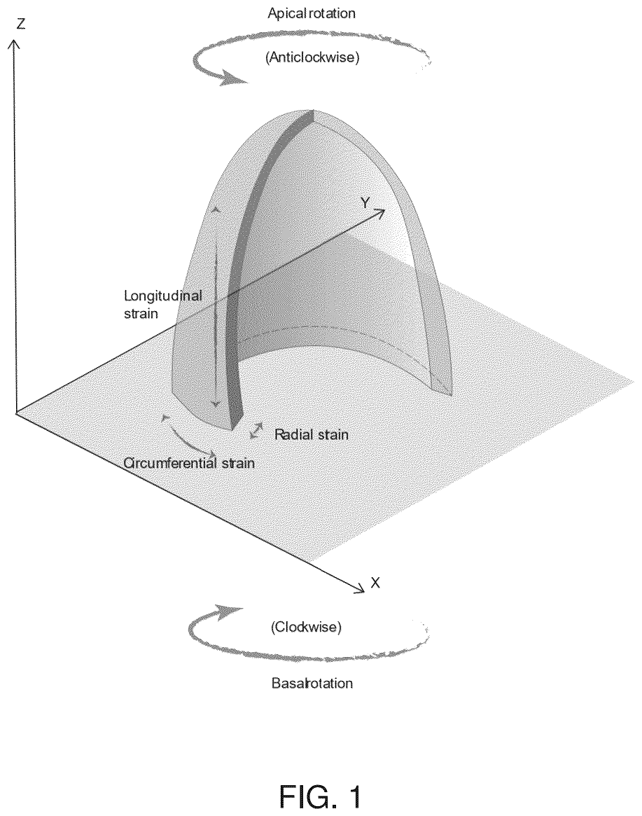

An example of the current state of the art for cardiac strain analysis, which analyzes at least one of the strain types shown in FIG. 1, relies on noninvasive calculations derived from multiple medical imaging modalities, primarily MRI, echo, and CT scan images. These technologies each present distinct advantages and limitations in clinical applications. MRI, while providing superior spatial resolution for detailed tissue analysis, is characterized by higher costs and longer acquisition times. In contrast, echocardiography offers enhanced temporal resolution and greater accessibility as a quick, economical option for obtaining clinical data on myocardial motion, but cannot provide measurements related to the thickness of the heart's chamber walls.

Commercial software packages employ several methodologies for strain analysis. Tissue doppler imaging, a fundamental approach, calculates strain by integrating velocity gradient over time, offering very good time resolution and fast qualitative analysis for local myocardial regions. However, it is constrained by its one-dimensional assessment capabilities and the requirement for ultrasound beam alignment. More advanced techniques include 2D and 3D speckle tracking, which track bright/dark interference patterns in cardiac tissue. While these methods allow strain calculation in any direction and enable torsion and area strain measurements, they are hampered by lower time resolution and significant post-processing dependencies.

MRI-based approaches encompass two primary methods: feature tracking and tagging. Feature tracking identifies and tracks individual anatomical features, enabling both local and global measurements, though local measurements show high variability. Tagging, which involves tracking imposed patterns of saturated myocardial magnetization, provides the most accurate local measurements but is limited by low temporal resolution, requires additional acquisition time, and involves demanding post-processing procedures.

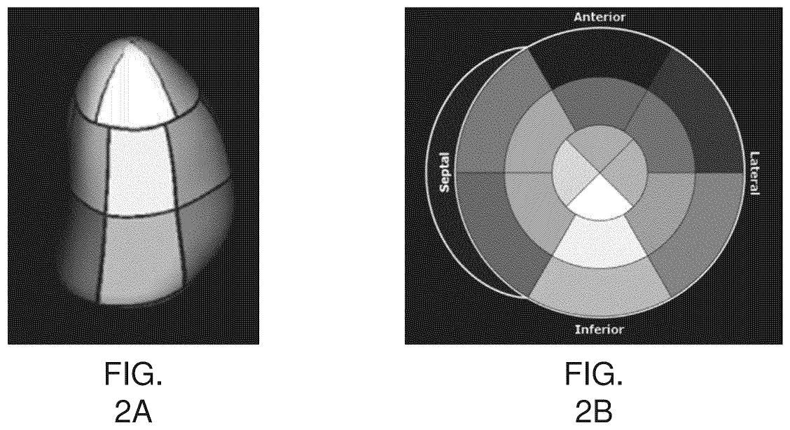

Current commercial platforms, such as 4D AutoLVQ (GE) and 4D LV-Analysis (TomTec), primarily focus on calculating three types of measurements: segmental strains, local one- or two-dimensional strains, and global strains. Some software packages derive radial strain calculations from area strain using volume conservation principles. A significant challenge in the field is the lack of standardization in strain calculation methods across different platforms and technologies, leading to potential inconsistencies in clinical assessments. Furthermore, the existing segmental approach typically involves dividing the chamber wall into large segments for strain measurements (e.g., as shown in FIGS. 2A and 2B), which limits the granularity of analysis.

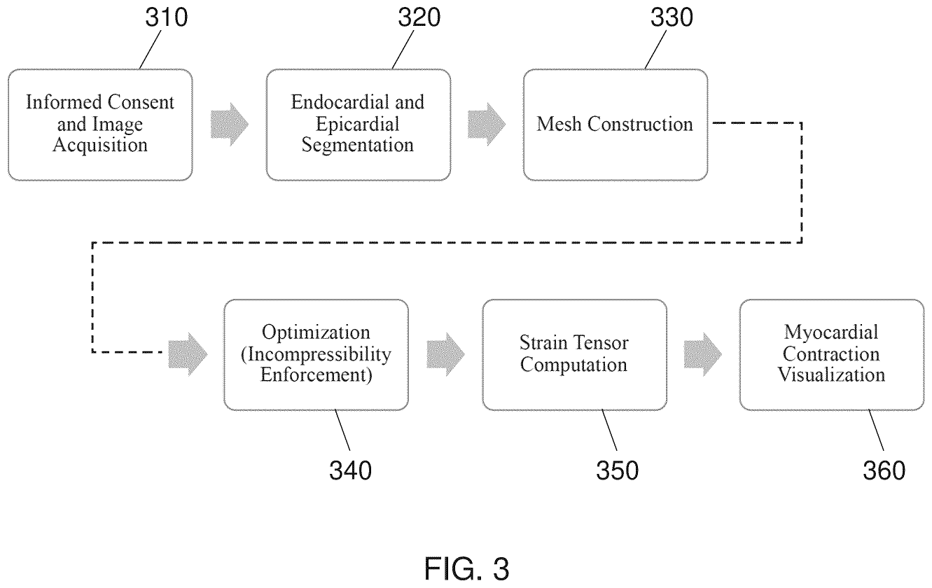

FIG. 3 is a schematic flowchart illustrating the sequential steps of an example strain analysis method. As shown therein, the strain analysis method begins with step 310 that includes obtaining patient consent and capturing four-dimensional digital images of the heart, e.g., echocardiographic image data, magnetic resonance imaging (MRI) data, or computed tomography (CT) data. In step 320, the process of extracting and delineating the inner and outer surfaces of the myocardial wall from the acquired imaging data is performed. The step 330 includes the segmented surfaces being used to construct a discrete tetrahedral mesh representation of the myocardium by combining the epicardial and endocardial triangulated surfaces. Then, in step 340, an optimization scheme is applied to refine the epicardial geometry and ensure that volumetric changes remain within physiological bounds consistent with the near-incompressible nature of myocardial tissue. This is followed by step 350 in which the calculation of the three-dimensional finite strain tensor for each tetrahedral element at each time step throughout the cardiac cycle is performed, e.g., using end-diastole as the reference configuration. Finally, at step 360, the computed strain data is visualized to characterize the myocardial deformation patterns, including the identification of principal strain directions and the reconstruction of functional fiber orientation networks.

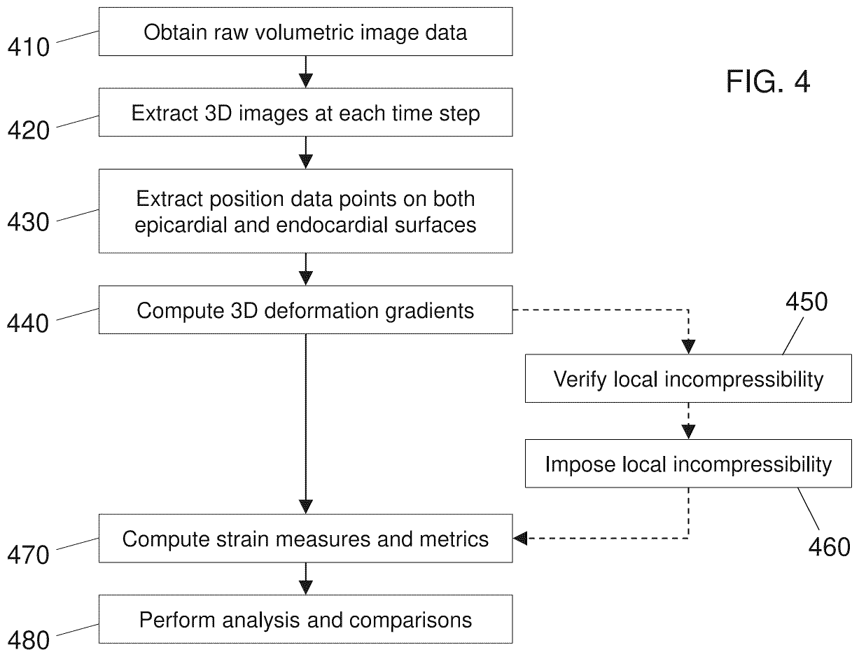

FIG. 4 illustrates a flowchart of a method of producing three-dimensional (3D) strain data associated with a heart in accordance with an example embodiment, which overcomes the drawbacks and limitations of existing methods and techniques for cardiac strain analysis. In some embodiments, producing 3D strain data includes measuring the strain associated with the heart (or a chamber thereof). In other embodiments, producing 3D strain data includes calculating the strain data (or one or more strain metrics) associated with the heart (or a chamber thereof).

As shown in FIG. 4, the method begins with step 410, where raw volumetric medical image data is obtained, e.g., through multiple imaging modalities including, but not limited to, MRI, echo, or CT scan. The raw volumetric data captured in this step contains information about the myocardial motion, which includes the endocardial and epicardial surfaces throughout the cardiac cycle. The data obtained is of sufficient quality to allow for the extraction of three-dimensional images at each time step of the cardiac cycle, and captures the motion of both the inner and outer layers of the cardiac chambers (e.g., left or right ventricle). For example, according to the disclosed embodiments, medical images can be processed in which the inner (endocardium) and outer (epicardium) boundaries of the myocardial wall are clearly visible. If part of the outer wall is outside the image frame, commercial software may generate segmentation results, but these are often unreliable. Similarly, unclear or blurry endocardial or epicardial borders during the cardiac cycle often lead to inaccurate segmentation. In such cases, expert cardiologists can assess the accuracy of the segmentation, though they typically recommend retaking the images.

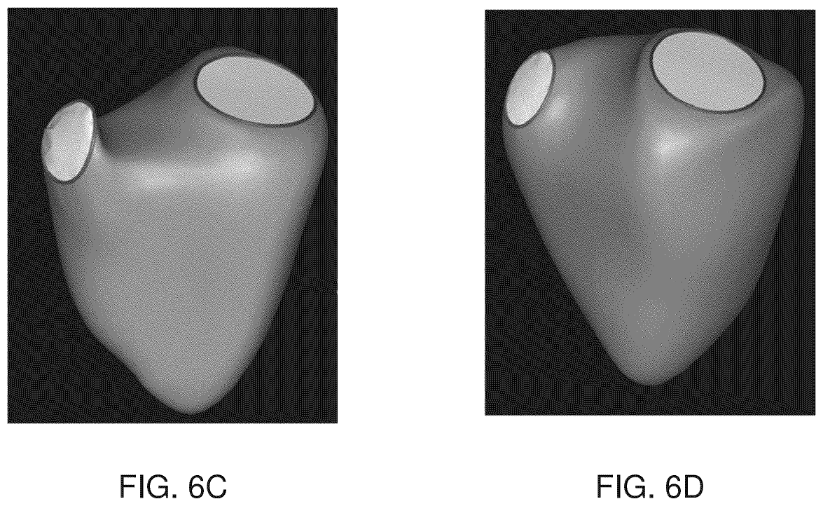

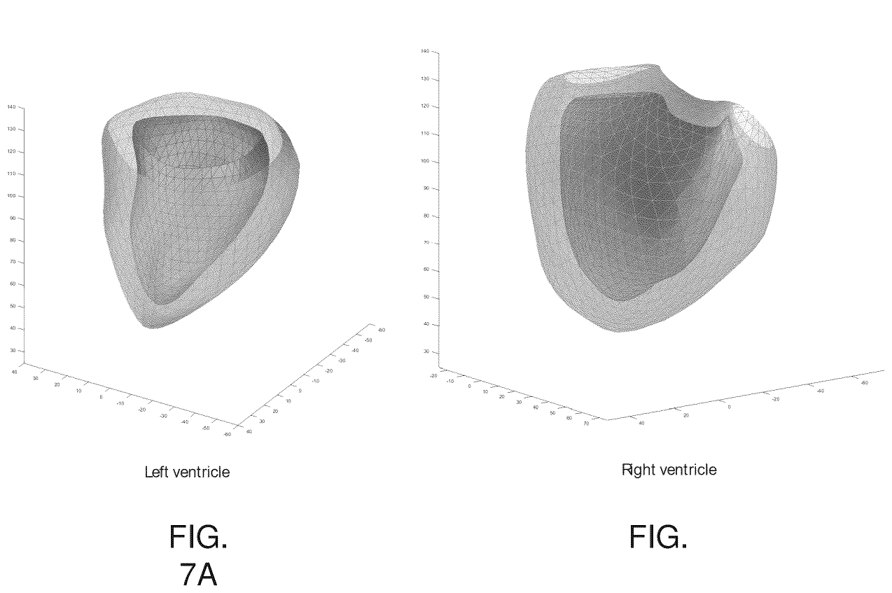

In step 420, a computer is used to extract three-dimensional images at each time step of the cardiac cycle from the raw volumetric medical image data obtained in step 410. Examples of the 3D images, for representative endocardial and epicardial surfaces of the left and right ventricles, are shown in FIGS. 6A-6D. The resolution of the three-dimensional images extracted in this step ensures the performance of the identification and tracking of the corresponding vertices on the endocardial and epicardial surfaces that are used for calculating the deformation gradients and strain measurements. In some examples, commercial software packages used for cardiac surface extraction and segmentation, which have a default spatial resolution and a fixed number of LV and RV points, generate triangular surfaces, which are processed by embodiments of the disclosed technology. In other examples, verifying or adjusting markers (e.g., valve locations and apex) or refining positions (e.g., valve leaflet positions) can be performed either before or after the generation of the triangulated surface by the commercial software packages.

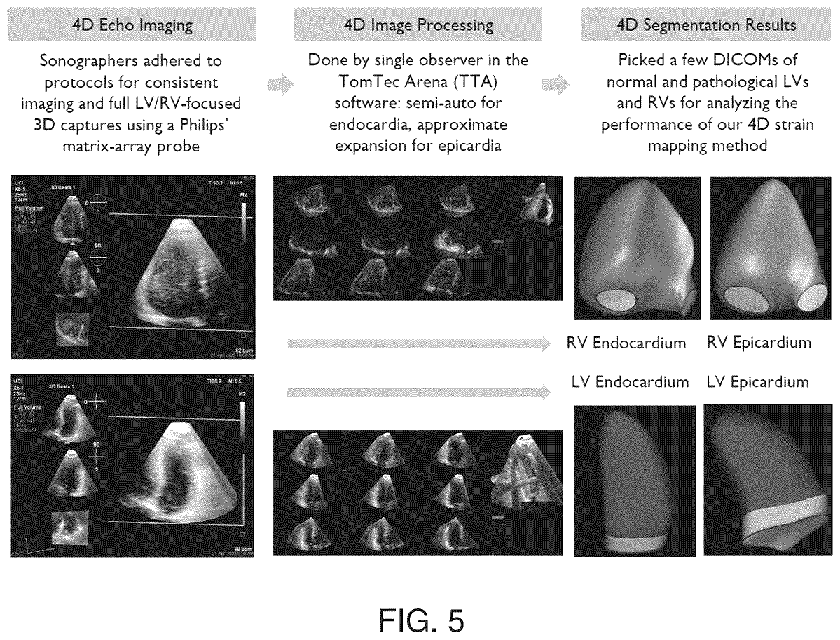

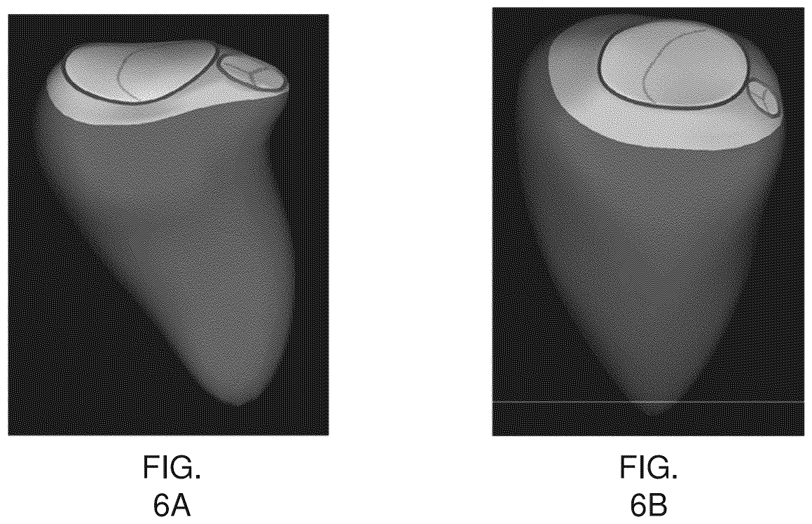

In some embodiments, post-processing of 4D digital image acquisitions may be performed using software to segment cardiac chambers and extract myocardial surfaces. FIG. 5 illustrates an example workflow comprising three stages. In a first stage (4D echo imaging), the initial acquisition includes LV-focused and RV-focused captures using a matrix-array probe. In a second stage (4D image processing), a 4D-LV-Analysis tool automatically generates a dynamic LV surface model and identifies end-systolic (ES) and end-diastolic (ED) frames, with optional manual refinement of contours. Similarly, a 4D-RV-Function tool may semi-automatically construct an RV model, requiring manual placement of landmarks such as the RV apex and tricuspid valve. Both tools may utilize 3D speckle-tracking optimized for capturing myocardial tissue motion across the cardiac cycle. This stage includes semi-automatic segmentation for endocardial surfaces and approximate expansion for epicardial surfaces. In a third stage (4D segmentation results), the final three-dimensional triangulated surface models for both LV and RV endocardium and epicardium are generated.

In some embodiments, epicardial delineation may be achieved by extending the endocardial segmentation functionality. For the LV, the software may provide an automated expansion of the endocardial contours to encompass visible myocardial tissue at ED. For the RV, this expansion may be performed manually. After manual adjustment of ED contours, the surface correction may be propagated across the cardiac cycle using built-in speckle tracking. The resulting ES contours may then be reviewed and manually edited as needed to ensure anatomical accuracy across all views.

In some embodiments, segmentation of echocardiographic images is inherently subject to inter-operator, intra-operator, and cross-platform variability, each of which can influence the accuracy and reproducibility of myocardial strain analysis. Inter-operator variability arises from differences in how individual observers delineate cardiac boundaries. Intra-operator variability reflects inconsistencies in repeated segmentations by the same observer. Cross-platform variability stems from differences in ultrasound hardware, image reconstruction algorithms, and post-processing software across vendors.

In some embodiments, compared to the endocardium, which is generally well visualized due to the distinct contrast between the blood pool and myocardial tissue, the epicardium often exhibits lower contrast, acoustic shadowing, and occasional dropout. Consequently, epicardial segmentation demonstrates substantially higher user- and platform-dependent variability than endocardial segmentation.

In some embodiments, the sensitivity of strain measures to epicardial segmentation variability may be evaluated by applying random perturbations to epicardial surface vertices. The endocardial ALS and ACS—which closely approximate clinically derived GLS and GCS—may be minimally affected by epicardial perturbations, indicating the robustness of these measures to small segmentation variations.

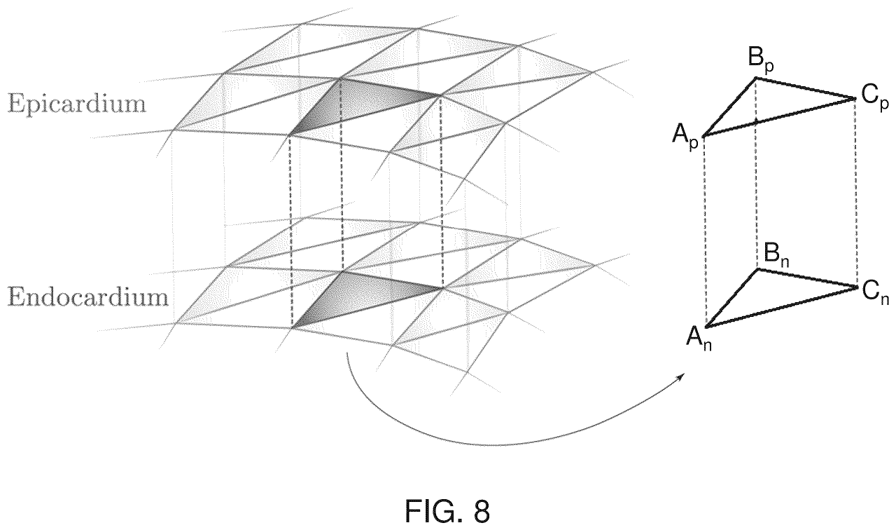

Step 430 in FIG. 4 involves extracting position data points from both the inner (endocardial) and outer (epicardial) surfaces of the cardiac chambers throughout the cardiac cycle. In some embodiments, three-dimensional echocardiographic strain for both ventricles is computed by constructing a discrete myocardial shell from triangulated epicardial and endocardial surfaces. At each time step of the cardiac cycle, the deformation gradient defines the local kinematics, enabling precise assessment of myocardial strain. This approach captures both normal and shear components while enforcing near-incompressibility to ensure a physiologically realistic representation of cardiac motion. In some embodiments, the cardiac cycle is divided into multiple time steps, with periodicity enforced such that the geometry at the final time step matches that at the initial time step. End diastole, when the ventricles are fully filled and fibers relaxed, is considered as the reference configuration. In some embodiments, both surfaces are overlaid with a triangular tessellation with identical connectivity between their vertices; an example of this operation is shown in FIGS. 7A and 7B for the left and right ventricles, respectively. In other embodiments, and more generally, a polygon tessellation can be overlaid on each of the surfaces with identical vertex connectivity. This operation creates a precise structural framework where each point on the inner surface has a corresponding point on the outer surface, establishing a one-to-one correspondence between vertices. The system tracks each vertex position throughout all time steps of the cardiac cycle, ensuring continuous monitoring of cardiac motion. For the case of the triangular tessellation, FIG. 8 shows the connectivity between a single triangle of the triangular tessellation on the endocardial surface and a corresponding triangle on the epicardial surface (and that forms an octahedron with the diagonals connected). As shown therein, the triangle on the epicardial surface with vertexes Aₚ, Bₚ and Cₚ corresponds to the triangle on the endocardial surface with vertexes Aₙ, Bₙ and Cₙ, respectively.

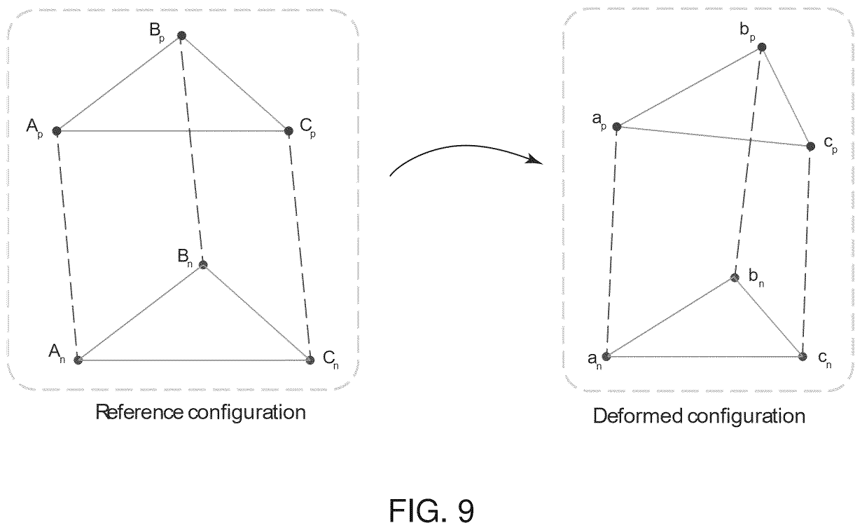

Step 440 involves computing the 3D deformation gradient using a selected time step as the reference configuration. In some embodiments, the connectivity of vertices on a triangulated myocardial surface generated by imaging software is identical for the epicardium and endocardium, establishing a bijection between the two meshes. This correspondence is used to construct a tetrahedral tessellation of the referential myocardial wall. The computational process starts with a octahedron element (Aₙ, Bₙ, Cₙ, Aₚ, Bₚ, Cₚ), where (Aₙ, Bₙ, Cₙ) represents a triangle on the tessellated endocardium and (Aₚ, Bₚ, Cₚ) is its corresponding triangle on the tessellated epicardium. It should be noted that the term octahedron is used herein to describe the geometrical feature with a volume (e.g., a polyhedron) that is enclosed between a polygon on the endocardial surface and the corresponding polygon on the epicardial surface. During deformation, and as illustrated in FIG. 9, these points in the reference configuration (Aₙ, Bₙ, Cₙ, Aₚ, Bₚ, Cₚ) are mapped to their corresponding positions (aₙ, bₙ, Cₙ, aₚ, bₚ, Cₚ) in the deformed configuration. In some embodiments, the deformation map satisfies periodicity over the cardiac cycle. The Jacobian of the deformation (J=det F) represents the ratio of the deformed volume to the reference volume of a tetrahedron.

In some embodiments, each octahedron is partitioned into three tetrahedrons. For the octahedra to completely cover the entire myocardium without any gaps, every pair of adjacent octahedra must interlock seamlessly. Adjacent octahedra that interlock seamlessly share two triangular faces and five edges with the Hadamard compatibility conditions holding across the shared faces. Herein, Hadamard compatibility refers to the rank-1 compatibility condition that restricts how deformation gradients or velocity gradients jump across a discontinuity surface (like a material interface) to be a tensor of rank 1.

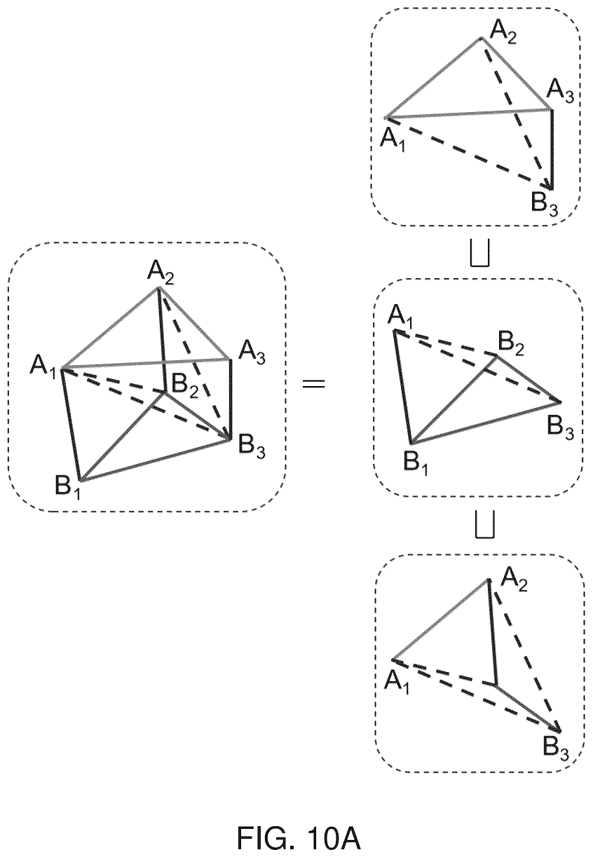

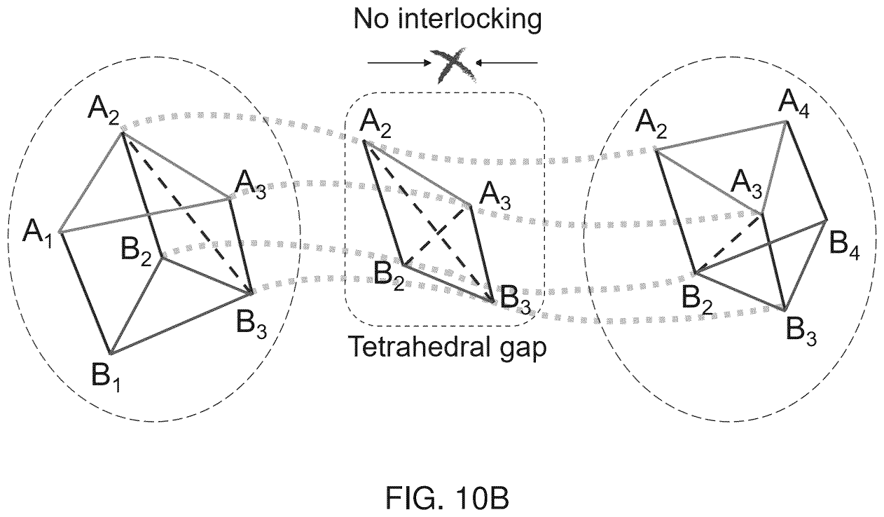

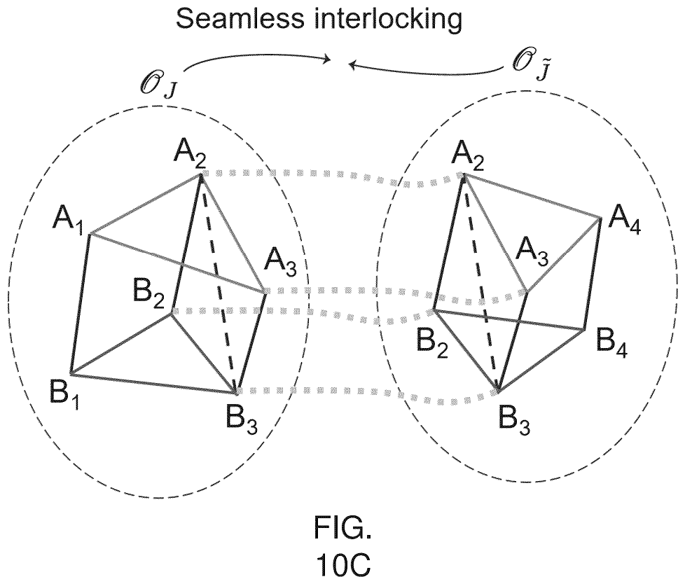

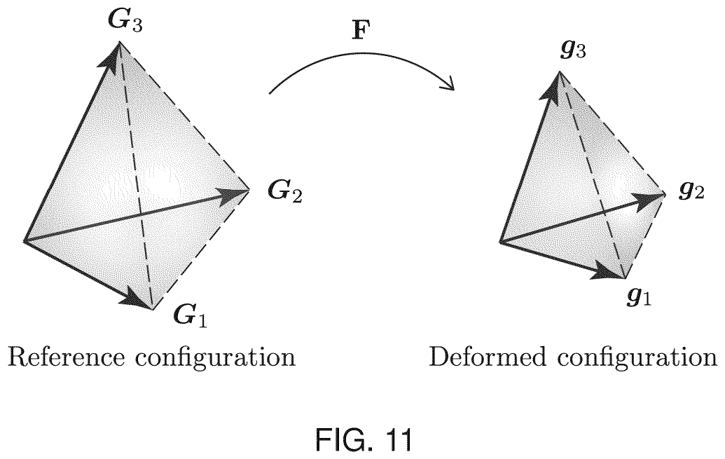

As shown in FIG. 10A, the octahedron element (Aₙ, Bₙ, Cₙ, Aₚ, Bₚ, Cₚ) is divided into three distinct tetrahedra: (Aₙ, Bₙ, Cₙ, Aₚ) that is used for endocardial strain calculations, (Aₚ, Bₚ, Cₚ, Bₙ) that is used for epicardial strain calculations, and (Bₙ, Cₙ, Aₚ, Cₚ) that is used for myocardial strain calculations. This specific partitioning, while not unique, provides comprehensive coverage of the myocardial region. Other examples of partitioning an octahedron into three tetrahedrons can be implemented without leaving gaps between adjacent octahedrons. FIG. 10B shows a partitioning example that results in a tetrahedral gap between adjacent octahedrons that do not interlock, and thus do not provide optimal results. In contrast, the partitioning example in FIG. 10C results in interlocking adjacent octahedrons without gaps.

One example method to avoid tetrahedral gaps between adjacent octahedrons begins with ordering the vertex indices for each triangle on each surface, and then ensuring that the smaller index vertex on the epicardium is connected to the greater index vertex on the endocardium when making the diagonal connections. Other methods that ensure seamless interlocking of adjacent octahedrons also exist.

The deformation gradient calculation for each tetrahedron includes, as shown in FIG. 11, analyzing edge vectors sharing a common vertex. Taking the tetrahedron (Aₙ, Bₙ, Cₙ, Aₚ) as an example, three edge vectors (G₁, G₂, G₃) are defined in the reference configuration: G₁=Bₙ−Aₙ, G₂=Cₙ−Aₙ, and G₃=Aₚ−Aₙ, with Aₙ serving as the origin. The corresponding edge vectors (g₁, g₂, g₃) in the deformed configuration are g₁=bₙ−aₙ, g₂=Cₙ−aₙ, and g₃=aₚ−aₙ. The deformation gradient F satisfies gᵢ=F Gᵢ for each tetrahedron, meaning F is a linear transformation that maps the reference edges to the respective edges of the deformed tetrahedron. The deformation gradient is uniquely defined for a given tetrahedron. The deformation gradient F is then calculated using these vectors and their dual basis through the following equation:

$$F = {g}_{1} ⊗ {G}^{1} + {g}_{2} ⊗ {G}^{2} + {g}_{3} ⊗ {G}^{3} .$$

Herein, ⊗ represents the tensor product of vectors in [symbol] ³, and the dual basis {G₁, G₂, G₃} corresponding to the reference edge vectors {G₁, G₂, G₃} is defined through the following relation:

$${G}_{i} \cdot {G}^{j} = \{ \begin{matrix}1 , & i = j \\ 0 , & i \neq j\end{matrix} .$$

In some embodiments, Steps 5 and 6 can be optionally implemented to verify and impose (if necessary), local incompressibility constraints that ensure that the volume of each tetrahedron is preserved during each time step of the cardiac cycle.

Step 450 involves verifying the incompressibility constraint for each time step by using the computed 3D deformation gradient to ensure that the volume of each tetrahedron is preserved during each time step of the cardiac cycle. This constraint can be mathematically expressed as:

J=det F=1.

Alternatively, this constraint can be expressed as:

$$J = \frac{[ {g}_{1} {g}_{2} {g}_{3} ]}{[ {G}_{1} {G}_{2} {G}_{3} ]} .$$

Herein, [u ν w]=u·(ν×w).

Step 460 enforces the local incompressibility constraint by perturbing the extracted points on the epicardial surface and/or the endocardial surface. In some embodiments, only the extracted points on the epicardial surface are perturbed, as perturbing both surfaces simultaneously may introduce locking artifacts. Restricting perturbations to the epicardial surface avoids such artifacts while still achieving the incompressibility objective, and is also preferred because endocardial segmentation is typically more reliable due to its higher contrast with the blood pool. In some embodiments, the perturbation (Su) is performed such that the following error (E) is minimized:

$$E = \sum {( J - 1 )}^{2} + P .$$

Herein, P>0 is a penalty term that must be added to ensure smoothness of the perturbed epicardium. In some examples, the following error expression is used for the optimization:

$$Error = \frac{1 - K}{{N}_{T}} {\sum }_{all octahedra} g ( J ) + \frac{K}{{N}_{V}} {\sum }_{all vertices} {‖ Δ ( δ u ) ‖}^{2} .$$

Herein, 0<K<1 is an optimization control parameter, N_{T} is the number of triangles on each surface, N_{V} is the number of vertices on each surface, and g(J)≥0 is a smooth function that satisfies g(1)=0. Furthermore, ∥Δ(δu)∥ denotes the discrete Laplacian norm of the perturbation of a vertex V that is perturbed by δu.

In some embodiments, the averaging summation in the “Error” equation above may implemented as a simple arithmetic mean or a weighted average that uses different volume-based weights and/or area-based weights.

The Laplacian operator quantifies the deviation of a function at a point from the average value over its local neighborhood, and minimizing its norm suppresses sharp, irregular features. In thin-shell structures, this Laplacian norm energy is analogous to the local bending energy, and its minimization mitigates abrupt curvature variations.

In the above, example choices for g(J) include the following:

$${g}_{1} ( J ) = {( J - 1 )}^{2}$$

$${g}_{2} ( J ) = {J}^{5} + \frac{1}{{J}^{5}} - 2$$

$${g}_{3} ( J ) = {( {J}^{2} - \frac{1}{{J}^{2}} )}^{2}$$

More generally, the function g(J) can take the following forms:

$$g ( J ) = {( {J}^{n} - 1 )}^{2 m}$$

$$g ( J ) = {( {J}^{p} + {J}^{- p} - 2 )}^{2 q}$$

$$g ( J ) = {( {J}^{α} - {J}^{- α} )}^{2 β}$$

$$g ( J ) = {( ln J )}^{\frac{2}{2 r - 1}}$$

Herein, m, n, p, q, α, β and r are positive integers.

In some embodiments, the optimization may be performed using the limited-memory Broyden-Fletcher-Goldfarb-Shanno (L-BFGS) algorithm. The penalty control parameter K may be varied to study its effect on convergence speed, Jacobian accuracy, and surface smoothness. Lower K values reduce the influence of the penalty term, allowing the solution to reach near-incompressibility with fewer iterations and faster convergence. In some examples, K values in the range of 0.1-0.25 may yield rapid convergence (typically within 20-25 iterations), while keeping Jacobian errors below 1% and maintaining visually smooth solutions.

The example method continues from either step 440 (if the optional incompressibility constraint is not verified and imposed) or step 460 (if the incompressibility constraint is imposed) to step 470, which involves calculating one or more strain metrics and/or principal directions. In the incompressibility-enforced myocardium, principal strains and directions for each tetrahedral element are obtained via Cholesky factorization of the symmetric strain tensor. The principal stretches are ordered as Δ₁≤λ₂≤₃. During systole, λ₁<λ₂<1, while the corresponding principal directions are approximately tangential to the myocardial surfaces, indicating in-plane contraction. In contrast, λ₃>1 and the corresponding direction is along wall thickness, reflecting systolic thickening. In some embodiments, the dominant contractile strain (λ₁−1) is distributed with good spatial uniformity over different regions, and the corresponding eigenvectors lie on surfaces surrounding the ventricle displaying spatial continuity. In contrast, the second principal strain (λ₂−1), which is lower in magnitude, presents more pronounced spatial variability in terms of both value and direction. The network of strain lines associated with the maximum contraction reflects the underlying myocardial fiber architecture or, to be precise, the net result of their contraction. The three principal directions that are computed provide insights into the local tissue deformation pattern. For example, two principal directions associated with negative strain are approximately tangential to the epicardial and endocardial surfaces, whereas the third principal direction is along the thickness of the myocardial layer.

In some embodiments, the strain metrics that are calculated include a strain tensor, an incremental strain, or a strain rate. The strain tensor calculated can be a Cauchy-Green-type strain tensor (e.g., the Cauchy-Green strain tensor), a Green-Lagrangian-type strain tensor (e.g., the Green-Lagrangian strain tensor), or an Eulerian-Almansi-type strain tensor (e.g., the Eulerian-Almansi strain tensor).

In some examples, the Cauchy-Green strain tensor is calculated as:

C=F^{T}F.

In some examples, the Green-Lagrangian strain tensor is calculated as:

$$E = \frac{1}{2} ( C - I ) .$$

In some embodiments, and as part of step 480, the eigenvalues and eigenvectors of the strain tensor (e.g., a 3×3 matrix) can calculated to determine the fiber direction in the myocardial layer. The deformation gradient F admits a polar decomposition F=QU=VQ, where Q is a unique orthogonal transformation, and both U (the right stretch tensor) and V (the left stretch tensor) are unique symmetric positive-definite tensors. The principal stretches (Qui) are the eigenvalues of the right stretch tensor U, and the quantities (λi−1) are referred to as the principal strains. The corresponding eigenvectors are the principal directions. Each principal direction in the reference configuration is mapped to a corresponding direction in the deformed configuration. In the example of the Cauchy-Green strain tensor, let

$${λ}_{1}^{2} , {λ}_{2}^{2} and {λ}_{3}^{2}$$

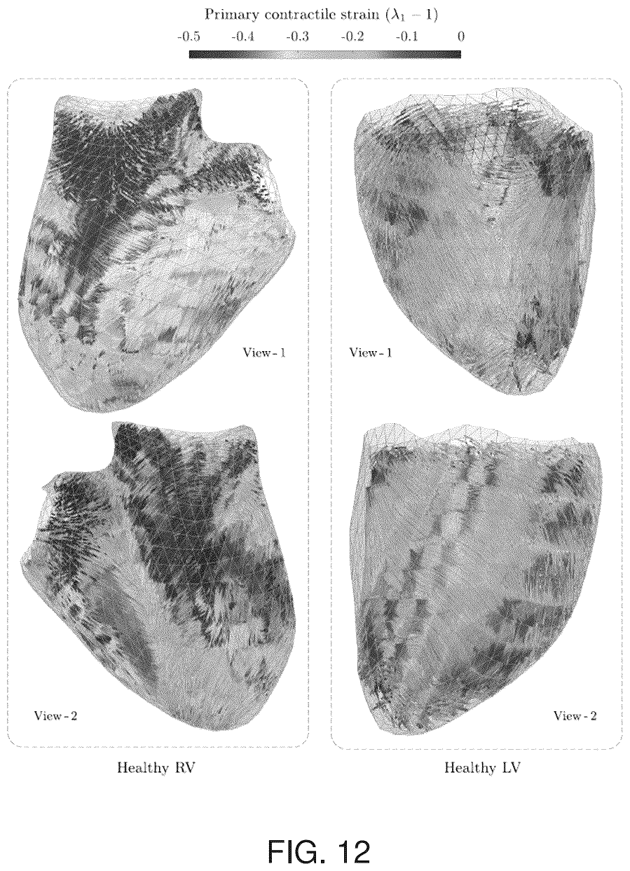

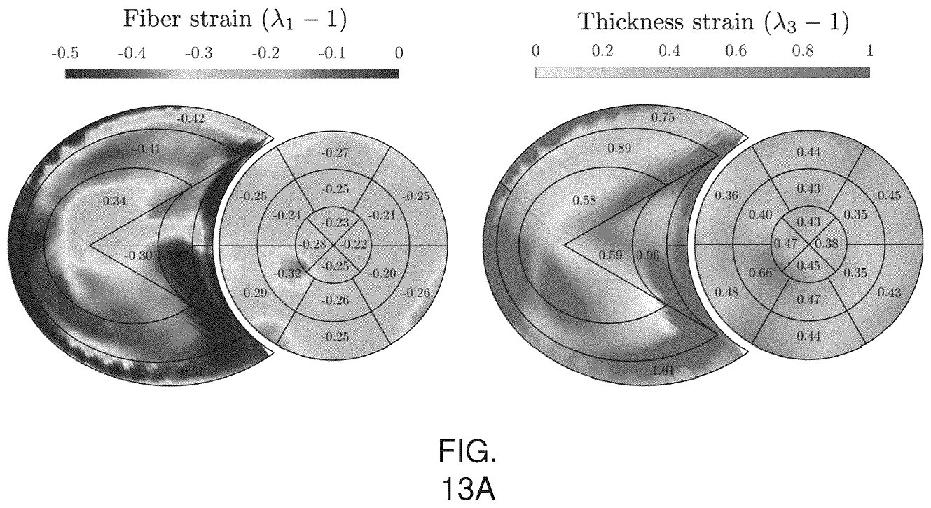

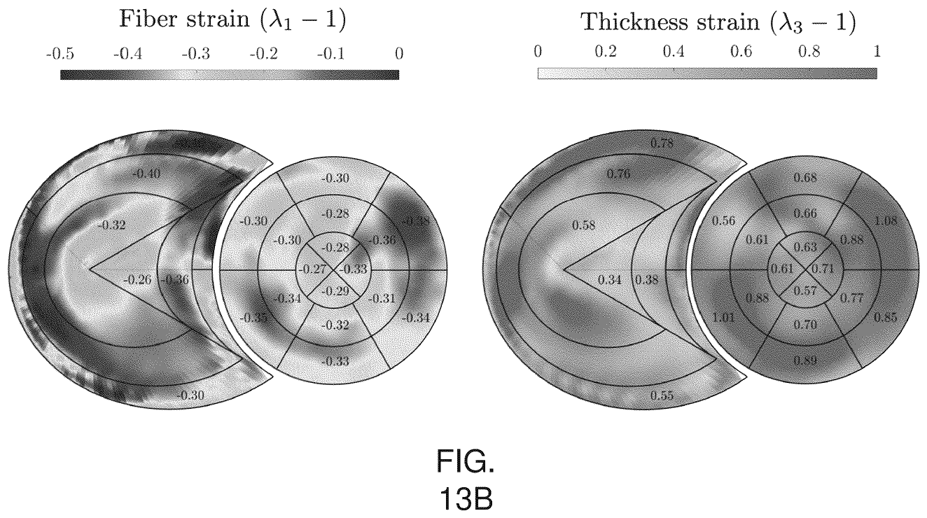

be the eigenvalues of C; ν₁, ν₂ and ν₃ be the corresponding eigenvectors; and λᵢ−1 represent the principal strain. To determine the fiber direction, the eigenvector that is closest to the normal direction of the surface is eliminated, and the remaining two eigenvectors correspond to negative principal strains. Then, the fiber direction is determined by identifying the eigenvector that corresponds to the most negative λ₁−1 value. In alternative embodiments, the eigenvector that corresponds to the maximum |ᵢ−1| value may be identified. It is noted that this approach aligns with the physical understanding that tangential strains, using the end-diastole as a reference, indicate contraction and align with the net result of muscle fiber orientation. FIG. 12 illustrates an example of visualizing the principal direction of the higher magnitude principal strain on representative left and right ventricle endocardium models. The color indicates principal strain magnitude (e.g., ranging from −50% strain to 0% (or no) strain), while the direction describes myocardial fiber function. While the use of eigenvectors to represent muscle fiber direction has been demonstrated before, the visualization is improved based on the disclosed embodiments that provide more comprehensive and accurate eigenvectors representative of cardiac function as compared to existing methods.

In some embodiments, the orientation of the dominant eigenvector representing the functional direction of fiber contraction may be characterized by the signed acute angle between the principal eigenvector and the circumferential direction in the short-axis plane. The circumferential direction is defined as the unit tangent to the myocardial surface in the short-axis plane, with orientation following the right-hand rule: when the thumb points from the apex toward the valves, the fingers curl along the circumference. Positive angles indicate that the principal direction forms an acute angle with the circumferential direction, tilting in the direction of the thumb (right-handed helix), whereas negative angles indicate a tilt in the opposite direction (left-handed helix). In some embodiments, in the LV endocardium, contraction directions are predominantly circumferential in the basal and mid-septal regions, shifting toward a more longitudinal orientation in other regions. The LV epicardium exhibits less circumferential and more oblique contraction patterns. The difference in primary contraction orientation between the endocardium and epicardium is more pronounced in the LV than in the RV. The RV shows relatively circumferential orientations in the septal region but transitions to a predominantly longitudinal pattern in the free wall, consistent with its greater base-to-apex displacement. The transmural variation of the principal contraction direction is more marked in the LV, reflecting its greater wall thickness, whereas the RV exhibits a comparatively uniform orientation across the wall.

In some embodiments, the disclosed embodiments provide comprehensive biventricular bullseye maps for intuitive visualization (e.g., of the strains computed as described above) and cohort-level comparison (e.g., comparing strain patterns across groups of subjects to identify characteristic patterns associated with different disease states). Bullseye plots visualize strain, volume-averaged over each octahedron, using colormaps. The segmental average strain values may be labeled within each segment. A 16-segment model is used for the LV, and a 6-segment model for the RV.

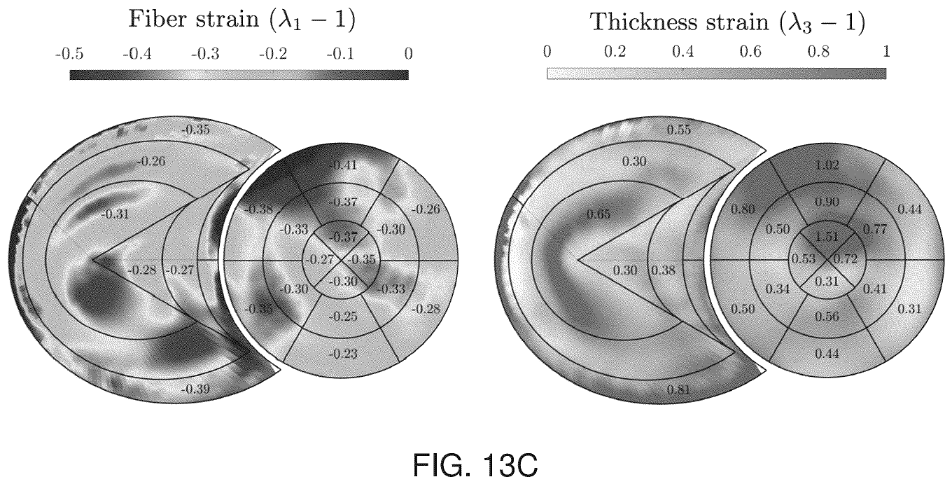

FIGS. 13A to 13C illustrates example bullseye visualization maps showing fiber strain (λ₁−1) (also referred to as primary contraction) and thickness strain (λ₃−1) as bullseye maps for both LV and RV for a healthy subject (shown in FIG. 13A), a subject with pulmonary hypertension and preserved ejection fraction (denoted PH-pEF, shown in FIG. 13B), and a subject with advanced pulmonary hypertension and reduced ejection fraction (denoted PH-rEF, shown in FIG. 13C). The combined RV-LV bullseye plots of fiber and thickness strains provide a convenient representation of the spatial deformation pattern, and simplify comparison across different subjects. This visualization format enables assessment of both the magnitude and spatial distribution of myocardial deformation, supporting the identification of regional dysfunction patterns that may not be apparent from global strain measurements alone.

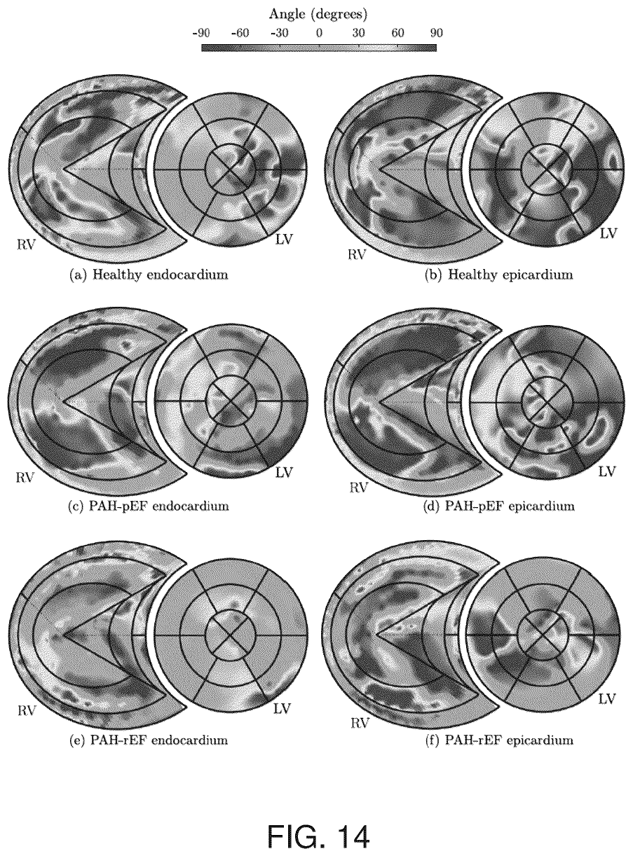

FIG. 14 illustrates biventricular bullseye maps showing primary contraction orientation for both endocardial and epicardial tetrahedra in healthy controls and patients with pulmonary hypertension. The orientation of primary (maximum) contraction is represented by the signed angle (from −90° to 90°) formed by the principal eigenvector relative to the circumferential direction in the short-axis plane. These bullseye maps enable visualization of transmural and regional variations in myocardial fiber contraction direction, supporting assessment of how disease progression affects the directional patterns of ventricular contraction.

Additionally, or alternatively, step 480 involves comparing components of the strain tensor and/or the eigenvalues between patients and healthy subjects, thereby providing a quantitative basis for identifying abnormal cardiac function.

In some embodiments, the direction of maximum contraction identified by the disclosed methods may be compared to myocardial fiber architecture imaged using other modalities, including diffusion tensor imaging (DTI). In DTI, the direction of maximal water diffusion is interpreted as the predominant orientation of cardiomyocytes. However, the fiber tracts reconstructed from diffusion MRI represent diffusion pathways rather than direct anatomical structures. Similarly, the direction of maximal contraction identified by the disclosed methods does not directly correspond to the true anatomical orientation of fiber function. Instead, it reflects the cumulative mechanical effect of all myofibers within the local neighborhood over which deformation is evaluated.

It is noted that the virtual fibers derived from both diffusion- and strain-based analyses do not represent discrete anatomical entities. Rather, they capture distinct yet complementary physical phenomena: anisotropic water diffusion along myocardial fibers in DTI, and localized contraction patterns measured through strain analysis. Together, these modalities offer an integrative framework that bridges structural and functional aspects of myocardial architecture.

In some embodiments, the described echocardiography-based strain analysis captures the temporal evolution of myocardial contraction and identifies the functional direction of deformation throughout the cardiac cycle. Given its lower cost, higher temporal resolution, and wider clinical accessibility compared to cardiac MRI, the disclosed embodiments enable practical, dynamic assessment of myocardial mechanics capable of detecting subtle functional alterations.

In some embodiments, the disclosed methods and systems may be applied to assess cardiac function in patients with pulmonary hypertension. Important cardiac dysfunctions-such as those associated with pulmonary hypertension-originate on the right side of the heart and primarily affect the right ventricle (RV) before impacting the LV. Application to healthy subjects and patients with pulmonary hypertension (PH) demonstrates attenuated right ventricular strain with compensatory enhancement of left ventricular deformation, underscoring its potential for early disease detection and mechanistic insight.

In some embodiments, analysis of patients with pulmonary hypertension and preserved ejection fraction (PH-pEF) may reveal characteristic strain patterns. The average LV strain curves for this patient present supernormal values in both longitudinal and circumferential components. Additionally, the time profiles of RV strain are altered with respect to the normal pattern, and present a slower systolic growth and absence of two well-recognizable phases of diastole. Regarding spatial distribution, the LV strain pattern indicates that the high average strain is not due to a uniform increase in contraction but is instead localized to a few specific regions. Consequently, this patient exhibits a loss of the uniform contraction typically seen in a normal LV. Spatial alterations in the strain pattern are also evident in the RV, which is more directly impacted by the disease. Despite normal average strain values, certain regions-such as the apex-show reduced strain.

In some embodiments, analysis of patients with advanced pulmonary hypertension and reduced ejection fraction (PH-rEF) accompanied by RV dysfunction may reveal different patterns. A marked reduction in ALS (below 10%) is observed when the contraction is averaged over the myocardium. Both LV contractile components are reduced, with a more pronounced decrease in the second principal strain, which is considered more sensitive to early dysfunction. In the RV, the average strain values show a greater reduction, reaching levels that can be considered pathological. The slight reduction in LV contraction comes at the cost of a spatial pattern that completely loses the uniformity typical of healthy contraction. The LV exhibits extensive regions of reduced strain interspersed with localized areas of elevated contraction, that are likely activated to compensate for the significant reductions. The RV presents a significant difference in the spatial pattern compared to both the normal RV and that of the PH-pEF patient, with a widespread reduction in contraction affecting nearly the entire myocardium. Additionally, the observed strain reductions are accompanied by a deviation in the principal direction of contraction toward a more circumferential orientation, particularly at the endocardial surfaces of both the LV and RV. As illustrated in FIGS. 13B and 13C, these changes correspond to disruptions in the regional strain patterns.

The ability to detect such variations in strain patterns across different disease stages may have clinical significance for earlier detection of cardiac dysfunction in its initial stages. In some embodiments, these findings suggest that this may be an attempt for the LV to increase its workload compensate for the right-heart dysfunction, and protect the weaker RV and allow to preserves its function in the early stage of the disease. The combined RV-LV bullseye plots of fiber and thickness strains in FIGS. 13A to 13C provide a convenient representation of the spatial deformation pattern and simplify comparison across different subjects. This comprehensive assessment of both the magnitude and spatial distribution of myocardial deformation may support improved diagnostic accuracy and monitoring of therapeutic response in patients with pulmonary hypertension.

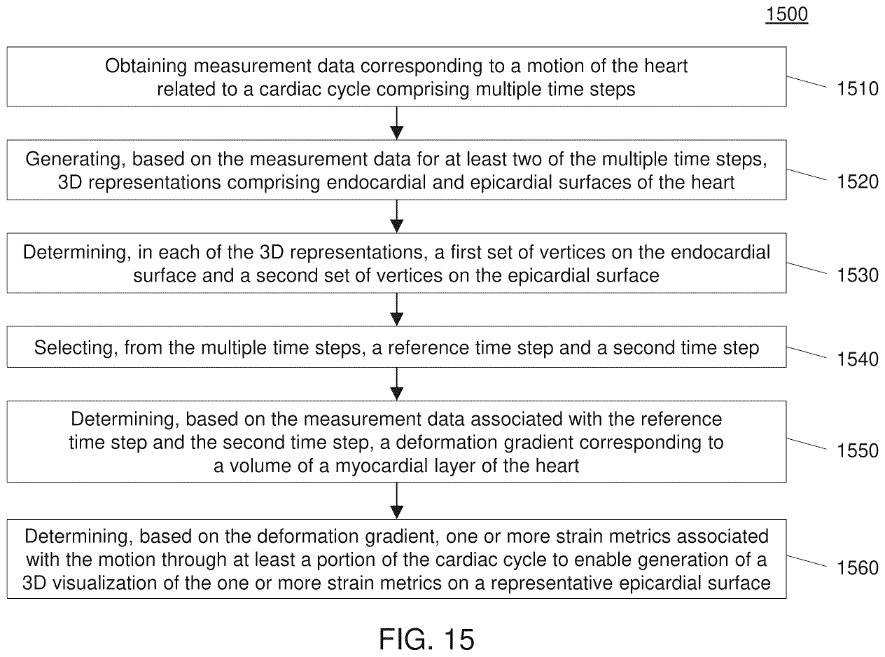

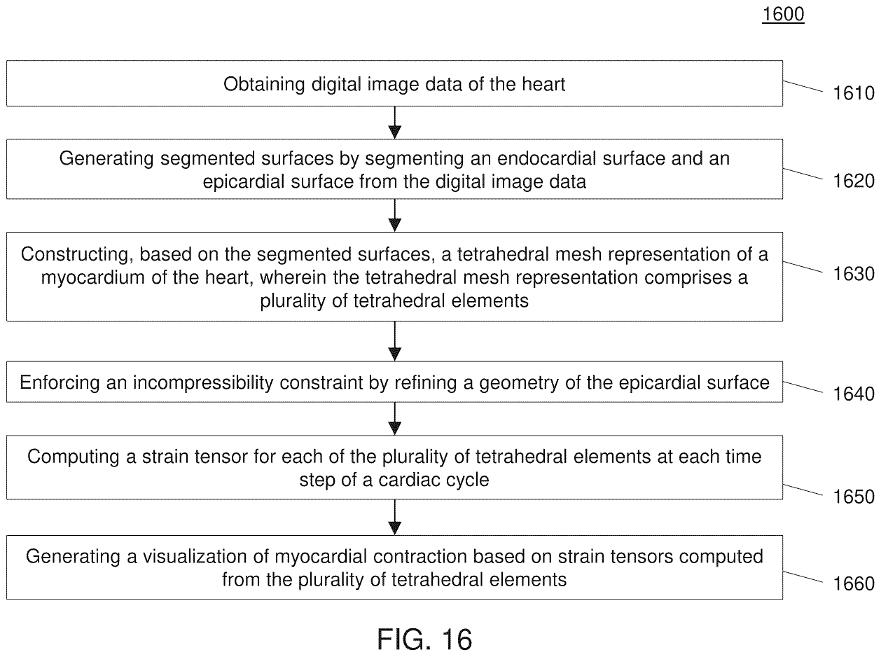

Embodiments of the disclosed technology are directed to and can be implemented through the following preferred technical solutions:
S1. A system for producing three-dimensional (3D) strain data associated with a heart chamber, comprising: one or more processors configured to: obtain measurement data corresponding to a motion of the heart chamber during a cardiac cycle comprising a plurality of time steps; for at least two of the plurality of time steps: generate, based on the measurement data, a plurality of 3D representations comprising an endocardial surface and an epicardial surface of the heart chamber, and determine, in each of the plurality of 3D representations, a first plurality of vertices on the endocardial surface and a second plurality of vertices on the epicardial surface, each of the first plurality of vertices corresponding to a respective vertex of the second plurality of vertices; select one of the plurality of time steps as a reference time step and another one of the plurality of time steps as a second time step; determine, based on the measurement data associated with the reference time step and the second time step, a deformation gradient corresponding to a volume of a myocardial layer of the heart chamber between a set of the first plurality of vertices and a respective set of the second plurality of vertices; and determine, based on the deformation gradient, one or more strain metrics associated with the motion through at least a portion of the cardiac cycle to enable generation of a 3D visualization of the one or more strain metrics on a representative epicardial or endocardial surface.
S2. The system of solution S1, wherein the one or more processors is configured to: determine that a local incompressibility constraint is satisfied based on the volume of the myocardial layer of the heart chamber being preserved across the plurality of time steps.
S3. The system of solution S1, wherein the one or more processors is configured to: determine that a local incompressibility constraint is not satisfied based on the volume of the myocardial layer of the heart chamber not being preserved across the plurality of time steps; perturb, for at least one time step, one or more vertices of the set of the first plurality of vertices or the set of the second plurality of vertices; and subsequent to perturbing the one or more vertices, determine an updated volume of the myocardial layer, and determine whether the local incompressibility constraint is satisfied based on the updated volume of the myocardial layer being preserved across the plurality of time steps.
S4. The system of solution S3, wherein the one or more processors is configured, as part of perturbing the one or more vertices, to: perturb only the one or more vertices of the set of the second plurality of vertices on the epicardial surface.
S5. The system of solution S3, wherein the one or more processors is configured, as part of perturbing the one or more vertices, to: minimize an error, for the at least one time step, between the volume of the myocardial layer in a reference configuration and a deformed configuration.
S6. The system of solution S5, wherein the error comprises a penalty term to ensure smoothness of the epicardial surface that includes the one or more vertices that were perturbed.
S7. The system of any of solutions S1 to S6, wherein the set of the first plurality of vertices and the respective set of the second plurality of vertices form a polyhedron.
S8. The system of solution S7, wherein the polyhedron is a octahedron, and the first plurality of vertices form a triangle.
S9. The system of any of solutions S1 to S8, wherein the one or more strain metrics comprises a strain tensor, an incremental strain, a strain rate, or associated eigenvalues and eigenvectors.
S10. The system of any of solutions S1 to S8, wherein the one or more strain metrics comprises a strain tensor, and wherein the strain tensor comprises at least one of a Cauchy-Green-type strain tensor, a Green-Lagrangian-type strain tensor, or an Eulerian-Almansi-type strain tensor.
S11. The system of any of solutions S1 to S8, wherein the one or more strain metrics comprises a strain tensor, and wherein the one or more processors is configured to: compute eigenvectors and eigenvalues of the strain tensor, wherein, between two eigenvectors corresponding to negative principal strains, a first eigenvector with a most negative eigenvalue is indicative of a direction of fibers in the volume of the myocardial layer.
S12. The system of solution S11, wherein the 3D visualization comprises plotting the first eigenvector on the representative epicardial or endocardial surface.
S13. The system of solution S11, wherein the one or more strain metrics comprises a strain tensor, and wherein the 3D visualization or a diagnostic analysis is enabled by a computability of each of nine components of the strain tensor.
S14. The system of any of solutions S1 to S13, wherein the measurement data comprises magnetic resonance imaging (MRI) data, echocardiography data, or computed tomography (CT) scan data.
S15. The system of any of solutions S1 to S14, wherein at least two of the plurality of time steps comprises more than two time steps encompassing an entire duration of the cardiac cycle, and wherein deformation gradients are determined based on the measurement data that corresponds to the plurality of time steps.
S16. The system of any of solutions S1 to S15, wherein the heart chamber is a left ventricle, and wherein generating the plurality of 3D representations is based on a representative shape of the left ventricle.
S17. The system of any of solutions S1 to S15, wherein the heart chamber is a right ventricle, and wherein generating the plurality of 3D representations is based on a representative shape of the right ventricle.
S18. The system of any of solutions S1 to S15, wherein the heart chamber is a left atrium, and wherein generating the plurality of 3D representations is based on a representative shape of the left atrium.
S19. The system of any of solutions S1 to S15, wherein the heart chamber is a right atrium, and wherein generating the plurality of 3D representations is based on a representative shape of the right atrium.
S20. The system of any of solutions S1 to S19, wherein the deformation gradient is determined for each time step between the reference time step and the second time step.
S21. A system of producing three-dimensional (3D) strain data associated with a heart, comprising: one or more processors configured to: obtain measurement data corresponding to a motion of the heart related to a cardiac cycle comprising a plurality of time steps; for a reference time step and a second time step of the plurality of time steps: generate, based on the measurement data, multiple 3D representations comprising an endocardial surface, a myocardial layer, and an epicardial surface of the heart, and determine, in each of the multiple 3D representations, a first tessellation on the endocardial surface and a second tessellation on the epicardial surface such that each first polygon of the first tessellation corresponds to a respective second polygon of the second tessellation; determine, based on the measurement data associated with the reference time step and the second time step, a first deformation metric for a first polyhedron, a second deformation metric for a second polyhedron, and a third deformation metric for third polyhedron; and determine, based on the first deformation metric, the second deformation metric, and the third deformation metric, one or more strain metrics associated with the motion through at least a portion of the cardiac cycle to enable a generation of a 3D visualization of the one or more strain metrics on a representative epicardial or endocardial surface.
S22. The system of solution S21, wherein the first polyhedron comprises at least three vertices from the first tessellation and at least one vertex from the second tessellation, the second polyhedron comprises at least one vertex from the first tessellation and at least three vertices from the second tessellation, and the third polyhedron comprises at least two vertices from the first tessellation and at least two vertices from the second tessellation.
S23. A method (e.g., method 1500 in FIG. 15) for producing three-dimensional (3D) strain data associated with a heart, comprising: obtaining (1510) measurement data corresponding to a motion of the heart related to a cardiac cycle comprising a plurality of time steps; for at least two of the plurality of time steps: generating (1520), based on the measurement data, a plurality of 3D representations comprising an endocardial surface and an epicardial surface of the heart, and determining (1530), in each of the plurality of 3D representations, a first plurality of vertices on the endocardial surface and a second plurality of vertices on the epicardial surface, each of the first plurality of vertices corresponding to a respective vertex of the second plurality of vertices; selecting (1540) one of the plurality of time steps as a reference time step and another one of the plurality of time steps as a second time step; determining (1550), based on the measurement data associated with the reference time step and the second time step, a deformation gradient corresponding to a volume of a myocardial layer of the heart between a set of the first plurality of vertices and a respective set of the second plurality of vertices; and determining (1560), based on the deformation gradient, one or more strain metrics associated with the motion through at least a portion of the cardiac cycle to enable generation of a 3D visualization of the one or more strain metrics on a representative epicardial surface.
S24. The method of solution S23, wherein generating the plurality of 3D representations comprises constructing a discrete myocardial shell from triangular tessellations on the endocardial surface and the epicardial surface, wherein connectivity of vertices on the triangular tessellations is identical for the endocardial surface and the epicardial surface, thereby establishing a bijection between the triangular tessellations.
S25. The method of solution S23, comprising: enforcing a periodicity such that a geometry of the endocardial surface or the epicardial surface at a final time step of the plurality of time steps matches the geometry at an initial time step of the plurality of time steps.
S26. The method of solution S23, wherein the first plurality of vertices and the second plurality of vertices form an octahedron, and wherein adjacent octahedra interlock seamlessly by sharing two triangular faces and five edges and connecting lower-index epicardial vertices to higher-index endocardial vertices.
S27. The method of solution S23, comprising: enforcing a local incompressibility constraint at each of the plurality of time steps using a limited-memory Broyden-Fletcher-Goldfarb-Shanno (L-BFGS) optimization algorithm.
S28. The method of solution S23, wherein the first plurality of vertices and the second plurality of vertices form a tetrahedral element, and wherein principal strains and directions for the tetrahedral element are obtained via Cholesky factorization of a symmetric strain tensor.
S29. The method of solution S23, wherein the one or more strain metrics is associated with a dominant eigenvector representing a functional direction of fiber contraction, and wherein an orientation of the dominant eigenvector is characterized by a signed acute angle between the dominant eigenvector and a circumferential direction in a short-axis plane.
S30. The method of solution S23, wherein the 3D visualization comprises a biventricular bullseye map for cohort-level comparison that includes comparing strain patterns across groups of subjects to identify characteristic patterns associated with different disease states.
S31. A method (e.g., method 1600 in FIG. 16) for producing three-dimensional (3D) strain data associated with a heart, comprising: obtaining (1610) digital image data of the heart; generating (1620) segmented surfaces by segmenting an endocardial surface and an epicardial surface from the digital image data; constructing (1630), based on the segmented surfaces, a tetrahedral mesh representation of a myocardium of the heart, wherein the tetrahedral mesh representation comprises a plurality of tetrahedral elements; enforcing (1640) an incompressibility constraint by refining a geometry of the epicardial surface; computing (1650) a strain tensor for each of the plurality of tetrahedral elements at each time step of a cardiac cycle; and generating (1660) a visualization of myocardial contraction based on strain tensors computed from the plurality of tetrahedral elements.
S32. The method of solution S31, wherein enforcing the incompressibility constraint comprises: determining that a local incompressibility constraint is not satisfied based on a volume of a myocardial layer not being preserved; perturbing one or more vertices on the epicardial surface; and determining an updated volume of the myocardial layer.
S33. The method of solution S31, wherein enforcing the incompressibility constraint comprises minimizing an error that includes a penalty term to ensure smoothness of the epicardial surface.
S34. The method of solution S31, wherein constructing the tetrahedral mesh comprises forming a plurality of octahedra, each octahedron comprising vertices from both the endocardial surface and the epicardial surface, and partitioning each octahedron into three tetrahedra.
S35. The method of solution S31, wherein the strain tensor comprises at least one of a Cauchy-Green-type strain tensor, a Green-Lagrangian-type strain tensor, or an Eulerian-Almansi-type strain tensor.
S36. The method of solution S31, comprising: computing eigenvectors and eigenvalues of the strain tensor; and determining a direction of fibers in the myocardium based on a first eigenvector corresponding to a most negative eigenvalue among eigenvectors corresponding to negative principal strains.
S37. The method of solution S31, wherein the digital image data comprises data obtained from a magnetic resonance imaging (MRI) scan, echocardiography, or a computed tomography (CT) scan.

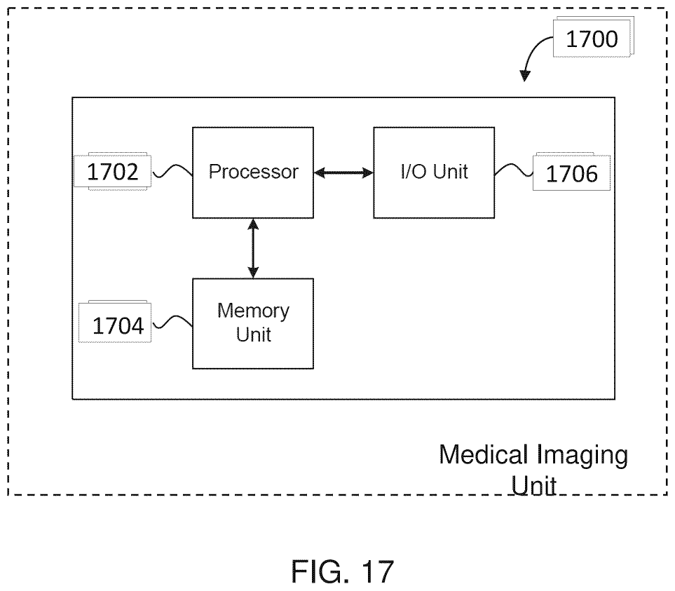

FIG. 17 shows a block diagram of an example embodiment of a device (or apparatus, hardware device or implementation) 1700 that implements the disclosed technology. The device includes a processor 1702 in communication with a memory unit 1704 and an input/output (I/O) unit 1706. The processor 1702 is configured to process data, and the memory unit 1704 is in communication with the processor to store and/or buffer the data. To support various functions of the device, the processor can be included to interface with and control operations of other devices, e.g., via the I/O unit 1706.

In some embodiments, and in the context of FIGS. 3-16, the processor 1702 can be configured to implement all or a portion of the generation of the 3D representation and the triangular tessellations, and the determination of the deformation gradients and one or more strain metrics of the methods described herein, and the I/O unit 1706 may be configured to receive the measurement data and visualize the strain metrics on a representative epicardial surface.

In various implementations, the processor 1702 can include one or more processors, e.g., including but not limited to microprocessors such as a central processing unit (CPU), microcontrollers, or the like. The memory unit 1704 can include and store processor-executable code, which when executed by the processor, configures the device to perform various operations, e.g., such as receiving information, commands, and/or data, processing information and data, and transmitting or providing information/data to another device. The memory unit can store other information and data, such as instructions, software, values, images, and other data processed or referenced by processor. For example, various types of Random Access Memory (RAM) devices, Read Only Memory (ROM) devices, Flash Memory devices, and other suitable storage media can be used to implement storage functions of memory unit. In some implementations, the device includes an input/output unit (I/O) 1706 to interface the processor and/or memory unit to other modules, units or devices associated with the system, and/or external devices. For example, the I/O unit can connect to an external interface, source of data storage, or display device. Various types of wired or wireless interfaces compatible with typical data communication standards, such as Universal Serial Bus (USB), IEEE 1394 (FireWire), Bluetooth, Bluetooth low energy (BLE), ZigBee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE cellular communication methods, and parallel interfaces, can be used to communicate data with the device via the I/O unit. In some implementations, for example, the device 1700 can include a wireless communications unit, e.g., such as a transmitter (Tx) or a transmitter/receiver (Tx/Rx) unit (not shown in FIG. 17). In such implementations, for example, the I/O unit can interface the processor and memory unit with the wireless communications unit to utilize various types of wireless interfaces, such as the examples described above. The I/O unit can interface with other external interfaces, sources of data storage, and/or visual or audio display devices, etc. to retrieve and transfer data and information that can be processed by the processor, stored in the memory unit, or exhibited on an output unit of a user device (e.g., display screen of a computing device) or an external device.

It is understood that the various disclosed embodiments may be implemented individually, or collectively, in devices comprised of electronic components, hardware and/or software modules and components. These devices, for example, may comprise a processor, a memory unit, an interface that are communicatively connected to each other, and may range from desktop and/or laptop computers, to mobile devices and the like. The processor and/or controller can be in communication with at least one memory and with at least one communication unit that enables the exchange of data and information, directly or indirectly, through the communication link with other entities, devices, and networks. The communication unit may provide wired and/or wireless communication capabilities in accordance with one or more communication protocols, and therefore it may comprise the proper transmitter/receiver antennas, circuitry, and ports, as well as the encoding/decoding capabilities that may be necessary for proper transmission and/or reception of data and other information.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described, and other implementations, enhancements and variations can be made based on what is described and illustrated in this patent document.

What is claimed is:

1. A system for producing three-dimensional (3D) strain data associated with a heart chamber, comprising:
one or more processors configured to:
obtain measurement data corresponding to a motion of the heart chamber during a cardiac cycle comprising a plurality of time steps;
for at least two of the plurality of time steps:
generate, based on the measurement data, a plurality of 3D representations comprising an endocardial surface and an epicardial surface of the heart chamber, and
determine, in each of the plurality of 3D representations, a first plurality of vertices on the endocardial surface and a second plurality of vertices on the epicardial surface, each of the first plurality of vertices corresponding to a respective vertex of the second plurality of vertices;
select one of the plurality of time steps as a reference time step and another one of the plurality of time steps as a second time step;
determine, based on the measurement data associated with the reference time step and the second time step, a deformation gradient corresponding to a volume of a myocardial layer of the heart chamber between a set of the first plurality of vertices and a respective set of the second plurality of vertices; and
determine, based on the deformation gradient, one or more strain metrics associated with the motion through at least a portion of the cardiac cycle to enable generation of a 3D visualization of the one or more strain metrics on a representative epicardial or endocardial surface.

2. The system of claim 1, wherein the one or more processors is configured to:
determine that a local incompressibility constraint is satisfied based on the volume of the myocardial layer of the heart chamber being preserved across the plurality of time steps.

3. The system of claim 1, wherein the one or more processors is configured to:
determine that a local incompressibility constraint is not satisfied based on the volume of the myocardial layer of the heart chamber not being preserved across the plurality of time steps;
perturb, for at least one time step, one or more vertices of the set of the first plurality of vertices or the set of the second plurality of vertices; and
subsequent to perturbing the one or more vertices,
determine an updated volume of the myocardial layer, and
determine whether the local incompressibility constraint is satisfied based on the updated volume of the myocardial layer being preserved across the plurality of time steps.

4. The system of claim 3, wherein the one or more processors is configured, as part of perturbing the one or more vertices, to:
perturb only the one or more vertices of the set of the second plurality of vertices on the epicardial surface.

5. The system of claim 3, wherein the one or more processors is configured, as part of perturbing the one or more vertices, to:
minimize an error, for the at least one time step, between the volume of the myocardial layer in a reference configuration and a deformed configuration, wherein the error comprises a penalty term to ensure smoothness of the epicardial surface that includes the one or more vertices that were perturbed.

6. The system of claim 1, wherein the first plurality of vertices form a triangle, wherein the set of the first plurality of vertices and the respective set of the second plurality of vertices form an octahedron.

7. The system of claim 1, wherein the one or more strain metrics comprises a strain tensor, an incremental strain, a strain rate, or associated eigenvalues and eigenvectors.

8. The system of claim 1, wherein the one or more strain metrics comprises a strain tensor, and wherein the one or more processors is configured to:
compute eigenvectors and eigenvalues of the strain tensor,
wherein, between two eigenvectors corresponding to negative principal strains, a first eigenvector with a most negative eigenvalue is indicative of a direction of fibers in the volume of the myocardial layer.

9. The system of claim 1, wherein the measurement data comprises magnetic resonance imaging (MRI) data, echocardiography data, or computed tomography (CT) scan data.

10. The system of claim 1, wherein the heart chamber is a right ventricle, a left ventricle, a right atrium, or a left atrium, and wherein generating the plurality of 3D representations is based on a representative shape of the right ventricle, the left ventricle, the right atrium, or the left atrium, respectively.

11. A method for producing three-dimensional (3D) strain data associated with a heart, comprising:
obtaining measurement data corresponding to a motion of the heart related to a cardiac cycle comprising a plurality of time steps;
for at least two of the plurality of time steps:
generating, based on the measurement data, a plurality of 3D representations comprising an endocardial surface and an epicardial surface of the heart, and
determining, in each of the plurality of 3D representations, a first plurality of vertices on the endocardial surface and a second plurality of vertices on the epicardial surface, each of the first plurality of vertices corresponding to a respective vertex of the second plurality of vertices;
selecting one of the plurality of time steps as a reference time step and another one of the plurality of time steps as a second time step;
determining, based on the measurement data associated with the reference time step and the second time step, a deformation gradient corresponding to a volume of a myocardial layer of the heart between a set of the first plurality of vertices and a respective set of the second plurality of vertices; and
determining, based on the deformation gradient, one or more strain metrics associated with the motion through at least a portion of the cardiac cycle to enable generation of a 3D visualization of the one or more strain metrics on a representative epicardial surface.

12. The method of claim 11, wherein generating the plurality of 3D representations comprises constructing a discrete myocardial shell from triangular tessellations on the endocardial surface and the epicardial surface, wherein connectivity of vertices on the triangular tessellations is identical for the endocardial surface and the epicardial surface.

13. The method of claim 11, comprising:
enforcing a local incompressibility constraint at each of the plurality of time steps using a limited-memory Broyden-Fletcher-Goldfarb-Shanno (L-BFGS) optimization algorithm.

14. The method of claim 11, wherein the one or more strain metrics is associated with a dominant eigenvector representing a functional direction of fiber contraction, and wherein an orientation of the dominant eigenvector is characterized by a signed acute angle between the dominant eigenvector and a circumferential direction in a short-axis plane.

15. A method for producing three-dimensional (3D) strain data associated with a heart, comprising:
obtaining digital image data of the heart;
generating segmented surfaces by segmenting an endocardial surface and an epicardial surface from the digital image data;
constructing, based on the segmented surfaces, a tetrahedral mesh representation of a myocardium of the heart, wherein the tetrahedral mesh representation comprises a plurality of tetrahedral elements;
enforcing an incompressibility constraint by refining a geometry of the epicardial surface;
computing a strain tensor for each of the plurality of tetrahedral elements at each time step of a cardiac cycle; and
generating a visualization of myocardial contraction based on strain tensors computed from the plurality of tetrahedral elements.

16. The method of claim 15, wherein enforcing the incompressibility constraint comprises:
determining that a local incompressibility constraint is not satisfied based on a volume of a myocardial layer not being preserved;
perturbing one or more vertices on the epicardial surface; and
determining an updated volume of the myocardial layer.

17. The method of claim 15, wherein enforcing the incompressibility constraint comprises minimizing an error that includes a penalty term to ensure smoothness of the epicardial surface.

18. The method of claim 15, wherein constructing the tetrahedral mesh representation comprises forming a plurality of octahedra, each octahedron comprising vertices from both the endocardial surface and the epicardial surface, and partitioning each octahedron into three tetrahedra.

19. The method of claim 15, wherein the strain tensor comprises at least one of a Cauchy-Green-type strain tensor, a Green-Lagrangian-type strain tensor, or an Eulerian-Almansi-type strain tensor, and wherein the digital image data comprises echocardiographic image data, magnetic resonance imaging (MRI) data, or computed tomography (CT) data.

20. The method of claim 15, comprising:
computing eigenvectors and eigenvalues of the strain tensor; and
determining a direction of fibers in the myocardium based on a first eigenvector corresponding to a most negative eigenvalue among eigenvectors corresponding to negative principal strains.
